(19) United States
(12) Patent Application Publication
(10) Pub. No.: US 2010/0292714 A1
(43) Pub. Date: Nov. 18, 2010

(54) SKIN MARKING DEVICE AND SUTURE HOLDER

(76) Inventors: Kaushal Shah, Forest Hills, NY (US); Joseph Habboushe, New York, NY (US)

Correspondence Address: SCHWEGMAN, LUNDBERG & WOESSNER/COLUMBIA UNIVERSITY, P.O. BOX 2938, MINNEAPOLIS, MN, 55402, US

(21) Appl. No.: 12/743,531

(22) PCT Filed: Nov. 18, 2008
(86) PCT No.: PCT/US08/12878

§ 371 Date: Jul. 28, 2010

Related U.S. Application Data
(60) Provisional application No. 61/003,636, filed on Nov. 19, 2007.

(51) Int. Cl. A61B 17/04; B05C 1/00

(52) U.S. Cl. 606/148; 118/255

(57) ABSTRACT

A skin marking device can include two or more skin marking members and a holding member coupled with the two or more skin marking members. The holding member can include at least one adjustment assembly. The adjustment assembly can adjust at least one of width or length between the skin marking members. In an example, a suture holder device can include a base and a member coupled with the base. The member can include one or more suture assembly holders. At least one attachment can be coupled with the base. The attachment can be adapted to couple the suture holder device with other structure.

### CLAIM OF PRIORITY

This patent application claims the benefit of priority, under 35 U.S.C. Section 119(e), to Kaushal Shah U.S. Provisional Patent Application Ser. No. 61/003,636, filed on Nov. 19, 2007 (Attorney Docket No. P21606).

### TECHNICAL BACKGROUND

In the medical arts, a patient's skin is marked to delineate an incision site, biopsy site, insertion point for needles or tubes and laceration repair. By marking the patient's skin doctors are able to ensure proper location of an incision during an operation and placement of sutures during wound closure procedures.

### SUMMARY

The present inventor has recognized, among other things, that sutures for wound closure should be placed equal distances apart, however, presently physicians have no means for precisely marking suture positions that are equidistant from one another. Rather, physicians are forced to use experience and judgment to determine proper placement of sutures. Suture needles that are inserted too close to a wound or incision edge may result in tearing when the area is stretched during normal activity by the patient. However, suture needles inserted too distant from a wound or incision edge may result in noticeable scarring. During the suture process, the needle of the suture is sometimes allowed to dangle while a knot is tied, which can lead to minor injury, needle sticks and non-sterile conditions.

This document describes, among other things, skin marking device and suture holder device and related methods. In some examples, a skin marking device can include two or more skin marking members, a holding member coupled with the two or more skin marking members, where the holding member can include at least one adjustment assembly, and the at least one adjustment assembly can adjust at least one of width or length between the skin marking members.

In other examples, a suture holder device can include a base, a member coupled with the base, where the member has one or more suture assembly holders. The member can be rotatably coupled relative to the base. At least one attachment can be coupled with the base. The attachment can be adapted to couple the suture holder device with other structure.

In other examples, a method of using a suture holder can include attaching a base of the suture holder device to a patient, the suture holder device including a member coupled with the base, the member having one or more suture assembly holders, at least one attachment coupled with the base, the attachment adapted to couple the suture holder device with other structure. The method can further include placing a suture assembly in the one or more suture assembly holders, and rotating the member and the one or more suture assembly holders relative to the base.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a schematic representation of an example of a skin marking device;

FIG. 2 illustrates a schematic representation of an example of a skin marking device;

FIG. 3 illustrates an example of usage of the skin marking device such as in accordance with the example shown in FIG. 2;

FIG. 4 illustrates a schematic representation of an example of a skin marking device and suture holder device;

FIG. 5 illustrates a perspective representation of an example of a suture holder device; and

FIG. 6 illustrates a side view of an example of a suture holder device.

### DETAILED DESCRIPTION

A skin marking device and a suture holder device and related methods, including various embodiments, are disclosed herein. The skin marking device allows skin to be marked in a systematic way in preparation for a medical procedure, e.g., laceration repair, marking the site of needle/tube insertion, biopsies, etc. This device can allow someone to accurately mark the skin for suture placement, for example, for wound closure. The skin can be marked for sutures to be placed equal distances apart. Some examples of the skin marking device would also allow for varying shapes (e.g., elliptical, tear-shaped, etc.) to mark the skin in preparation for excisional biopsies. The suture holder device can allow a suture needle to be secured when it is not needed, for example, while a knot is being tied, such as to avoid accidental needle sticks, and accidentally breaking sterility of the site. The suture holder device can further facilitate storage or tracking of needles, and loading of a needle into a needle driver.

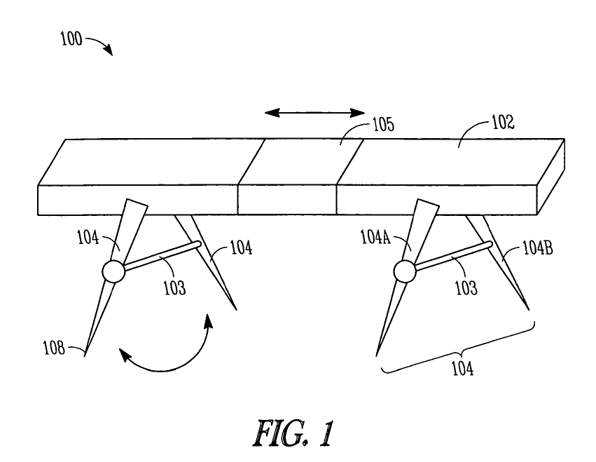

FIG. 1 shows an example of a skin marking device 100. In this example, the skin marking device 100 can include one or more skin marking members 104, such as two or more skin marking members 104. While FIG. 1 illustrates two sets of pairs of skin marking members 104, the skin marking device 100 can include more or less skin markings members 104, or pairs of skin marking members. In an example, the skin marking device 100 can be structured with a fixed length, and pairs of marking members 104 disposed at fixed intervals. The skin marking device 100 can be constructed from metal, plastic or any other material suitable for sterilization after use, or economical for disposal after a single use.

In an example, the skin marking device 100 can further include a holding member 102 coupled with the one, or two or more skin marking members 104. The holding member 102, in an example, can be used as a measuring tool where markings are provided on the holding member 102, such as 1 mm apart. The markings can indicate a measurement of separation between two marking members, either lengthwise or widthwise. In a further example, the holding member 102 can be formed of a flexible material so that irregular or non-linear paths can be marked on the skin, such as for accommodating lacerations of irregular size or shape.

In an example, the holding member 102 can includes at least one adjustment assembly 105, where the at least one adjustment assembly 105 adjusts, directly or indirectly, at least one of width or length between the skin marking members 104. In a further example, at least one adjustment assembly 105 can be operably coupled directly between a pair of skin marking members 104a, 104b.

The adjustment assembly 105 can be incrementally extended or shortened lengthwise or widthwise, in an example, and can also maintain a position of the skin marking members 104. This allows for the skin marking members 104 to be adjustable such as to accommodate various wound sizes. In an example, the adjustment assembly 105 can include a screw mechanism, such as a thumb screw, between two or more of the marking members 104. In another option, the adjustment assembly 105 can include a ratchet assembly. In a further example, the adjustment assembly 105 can include an electric motor, such as a piezoelectric motor, such as for precisely moving the marking members 104a and 104b and capable of holding the marking members securely in position.

In an example, the skin marking members 104 can be movably coupled with the holding member 102, so that the adjustment assembly 105 can move the skin marking members 104 into a desired position for marking the skin. In an example, the skin marking members 104 can be rotatably coupled with the holding member 102, such as a pair of opposed rotating marking members. In a further option, the skin marking members 104 can be folded away when not in used, such as to provide a compact shape for the device 100. In another example, the skin marking members 104 can be fixedly coupled with the holding member 102, and the adjustment assembly 105 can move and adjust the structure of the holding member 102.

The skin marking members 104, in an example, can include a dye 108 on or within the skin marking members 104. The dye can be disposed within a well within the skin marking members 104, or disposed on an outer portion of the skin marking members 104, for example. The dye 108 can be excreted upon contact with a patient's skin. Additionally, the marking dye 108 can be replaceable with different colors and different thicknesses. Moreover, the marking members can be adapted to provide a localized dosage of a numbing agent to reduce the pain resulting from the suturing process. Since the numbing agent is administered at the site of each marking rather than to the entire area, less numbing agent needs to be used, reducing the risk of toxicity. In addition or in alternative to using a skin marking dye, the skin marking members 104 can mark the skin with pressure indentation.

In using the skin marking device 100, the position of the skin marking members 104 can be adjusted, for example, with the adjustment assembly such as to place dye marks at specified separations, e.g., ⅛ inch (approximately 3 mm) apart. In addition or in alternative, the position of the skin marking members can be adjusted, in an example, so that the marks can be placed at the proper distance from the wound or incision edge. In this manner, the dye marks can delineate entry and exit points for the suture needle along the length of the wound.

Should the wound need more sutures beyond the number of marking members 104, the physician can generate additional dye marks on the patient's skin by spatially translating the skin marking device 100 along the length of the wound such that a pair of marking members 104 can be positioned such as to overlap the dye marks made by previous skin marking members 104. In this way the separation between dye marks can be uniformly maintained.

In an example of a method of use of the skin marking device, the device can be placed around a laceration, where the skin marking device includes a first skin marking member, and a second skin marking member, or more. The method can further include marking the skin with at least a first mark. The skin marking device can be lifted and moved over to overlap the second skin marking member over the first mark, and the first skin marking member is used to make a second mark. This method can be repeated around the laceration and provides equidistant suture markings.

In another example of a method for use of a skin marking device 100, for example, with four skin marking members 104, used for a laceration can include:

1) place device around one end of the laceration to make four marks;

2) lift the device and move it over such that two skin marking members, such as prongs, overlap on two marks and two new marks are made; and

3) continue in this manner until you reach the end of the laceration such that every two marks on either side of the laceration are equidistant from the next two marks on their side of the wound.

Excision biopsies are typically performed by elliptical incisions. When a six-prong device is used, the center skin marking members can be adjusted further apart and the peripheral skin marking members can be adjusted closer, such that a 3:1 elliptical shape is generated prior to making an incision. Once the skin is cut, the skin can be sutured and the device can again mark the precise location where the stitches should be inserted.

Physicians, veterinarians, medical students, professors, scientists, animal researchers, and health care workers, can use this device to mark the skin before treatment with incisions or suturing or other treatment. The device can be used to mark the skin for precise suture placement, to teach suture placement, to mark the skin for incision prior to cutting, mark the skin for excisions (e.g. 3:1 elliptical), to apply anesthetic in discrete locations with only minimal anesthetic use, or to assist in any or all combinations of the above.

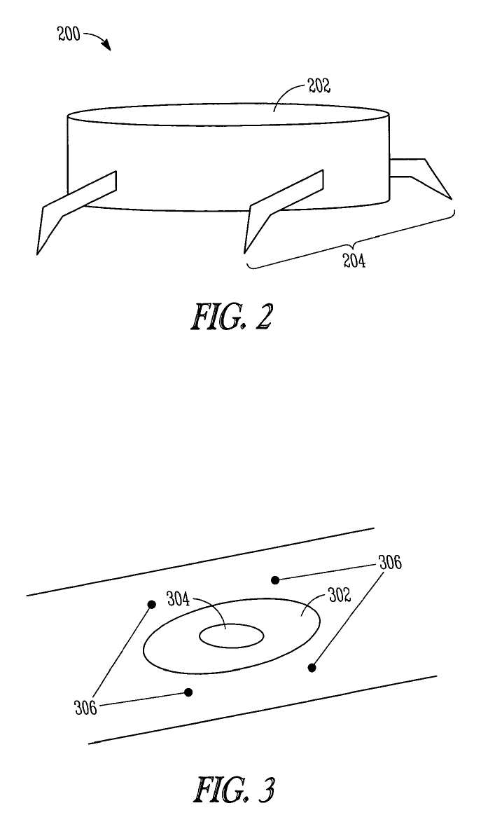

FIG. 2 shows another example of a skin marking. device 200. In this example, the device 200 can include an elliptical-shaped perimeter 202, with similar skin marking members 204 as described above for the example shown in FIG. 1. The device 200 can have other shapes, such as, but not limited to, circle, or rectangle. In an example, the skin marking members 204 can be sized appropriately for a specific size of the elliptical shaped skin marking device 200. In another example, the device 200 can include an adjustment assembly.

In an example, the elliptical-shaped perimeter 202 can include skin marking dye on a lower edge so that pressure applied to the skin with the elliptical skin marking device 200 transfers an identifiable elliptical mark 302 around the perimeter of the biopsy site 304. (FIG. 3). Markings 306 can be formed by skin marking members 204 and can be transferred at positions corresponding to suture positions as well. The top of the skin marking device can be covered by a translucent plastic or similar translucent material and provided with millimeter or inch or other measurement marks in either or both of the horizontal and vertical axis such that the physician can easily determine and confirm the distance from the biopsy site 304 to be for the excision/biopsy before making any marks or incisions.

In an option, a user can select a specifically sized skin marking device from a kit or set of multiple devices having different sizes. After the correctly-sized skin marking device is selected, the device 200 can be placed on the skin over the biopsy site 304 such that an ellipse 302 (marking the site of cutting/incision) and suture marks 306 are placed on the skin in the form of ink marks or indentation. The physician can then cut the skin along the elliptical mark 302 and can then place the sutures where the skin is already marked with the suture marks 306.

In an example, the elliptical device can include a sharp cuffing edge similar to a “punch biopsy” such that the skin is cut in the shape of an ellipse and the marking members 204 mark the skin for subsequent skin closure with sutures. In an example the elliptical device can include structure that cuts the skin, and brings two opposing sides together. For example, the member 204 can pierce the skin and the members 204 can be pressed and manipulated by the user, such as to bring the edges of the skin together.

The skin marking members can be of various or varying lengths and thicknesses, and can be modified such as to provide ink marking the skin, to provide indentation marking the skin, or to provide local anesthetic being injected or otherwise introduced in the skin. The skin marking members 204 can be a few millimeters high, such as when used for micro surgery, or few inches high, such as when marking a large wound or area (for example, on an elephant). In an example, the marking members can be removable and interchangeable, such that the skin marking device of either FIG. I or FIG. 2 can be customized by the physician such as to meet a specific need.

In an example, the skin marking device 200 can allow for precise marking of the skin before penetration with either sutures, needles, tubes, etc., can help in equally spacing stitches from the wound and from one another, can allow accurate elliptical incisions and wound closure, can help teaching medical students, physician assistants and resident where to place stitches, needles or tubes, can help in providing patients the benefit of seeing where and how many stitches will be needed or how large the incision will be before beginning the medical procedure, or can provide erasable marks if the projected suture sites are not acceptable for whatever reason. In an example, the device 200 can allow a preview of the final closure appearance before beginning an invasive procedure, preparation of wound closure (marking the future site of sutures) by less-trained personnel in preparation for wound closure by a physician, thus saving time for the physician, can allow precise excisions with a preview before cutting, and can facilitate anesthetic or antiseptic administration.

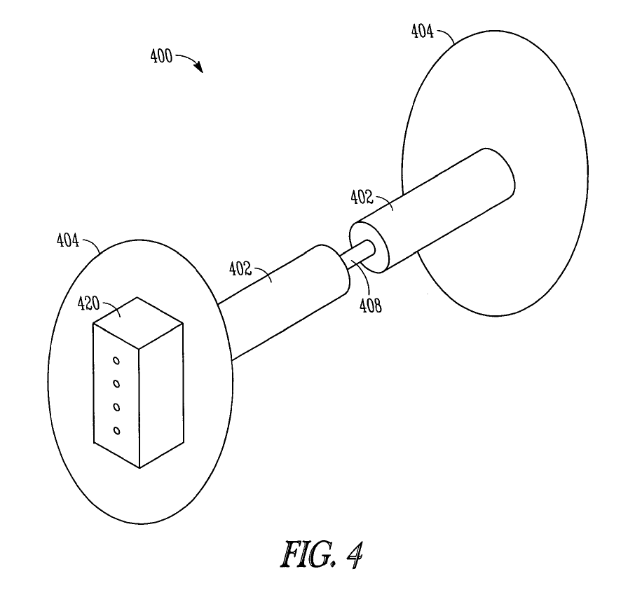

FIG. 4 shows an example of the skin marking device 400. In this example, the skin marking device 400 can include an optional suture holder device 420, two or more skin marking members 404, a holding member 402 coupled with the two or more skin marking members. In an example, the two or more skin marking members 404 have a circular shape and can be rotatably coupled with the holding member 402 on opposing portions of the holding member 402. In an example, the holding member 402 acts as an axle as the skin marking members 404 rotate. The skin marking members 404 can further include a dye or other type of skin marking material, in an example. The skin marking material can be disposed around a complete perimeter of the members 404, or can be discretely placed, so that a series of markings, or equally spaced markings can be made, in an example. In an example, the spacing between the markings can be adjustable either by adjusting the spacing mechanically or, alternatively, by replacing the marking ends with marking ends having the desired spacing.

In an example, the device 400 can further include an adjustment assembly 408. In an example, the adjustment assembly 408 can adjust a distance between the skin marking members 404, and can allow for the distance therebetween to be increased or decreased. In an option, the adjustment assembly 408 includes a screw that threadingly coupled with portions the holding member 402. In an example, the distance between the marking members 404 can be adjusted, such as by screwing or unscrewing the portions for the holding member 402. In an example, an indicator can be disposed along the holding member 402. This indicator can indicates the distance between the two skin marking members 404 in appropriate increments, such as millimeters, inches, etc.

During use of the device 400, the separation between the two skin marking members 404 can be adjusted, and the skin marking device 400 can be rolled along the wound, incision or biopsy site. As the skin marking device 400 rolls along the wound, the marking ends imprint markings onto the skin. These markings can form a solid line, or they can be a series of equally spaced dots.

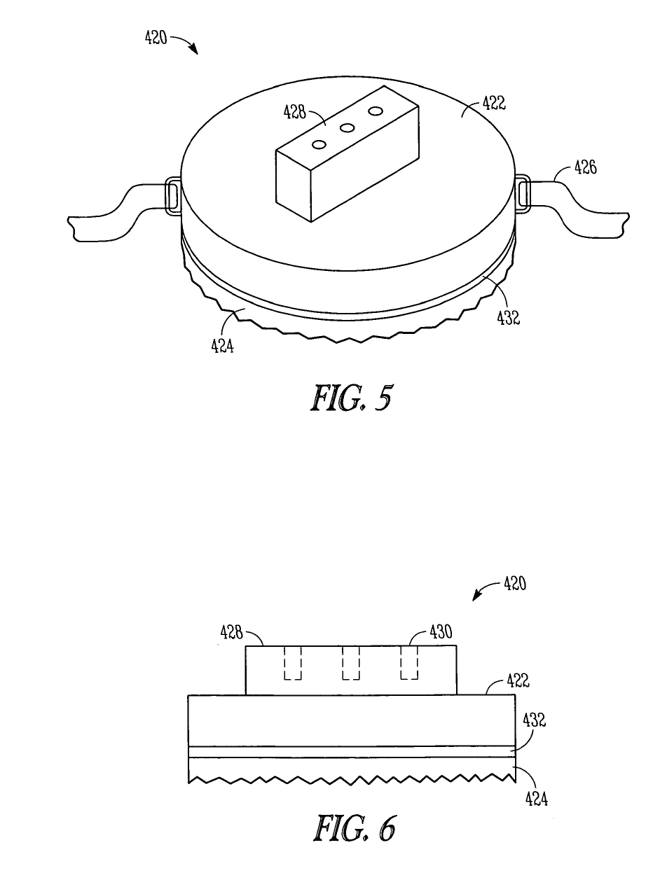

FIGS. 5 and 6 show examples of features and variations for the suture holder device 420. In an example, the suture holder device 420 can include a base 422, a member 428 coupled with the base 422. The member 428 can have one or more suture assembly holders that can be adapted to hold a suture assembly, in an example. The one or more suture assembly holders can be provided, in an example, by the member 428 being made of rubber, polymer, foam, plastic, metal or any such material that will allow a needle to be inserted and secured within. In an example, the member 428 can be magnetized or can include one or more sticky surfaces or materials such as to form the suture assembly holder. In another example, the suture assembly can include at least one recess 430 formed in the member 428 such as, for example, but not limited to, by pre-drilling one or more holes into the member 428. The various materials and recesses can be combined to have multiple ways to retain the suture assemblies. In an example, the member 428 can be sized or shaped, or both, to allow for the member 428 and optionally any needles therein to be disposed in a “sharps” container. In an example, the member 428 can be removable from the base 422. For example, the member 428 can include a magnetic or adhesive or other material or structure, which can allow for the member 428 to be removably coupled with the base 422. 100421 In an example, the suture holder device can include an attachment that can allows for the device 420 to be secured, for example, near the suture area. The attachment can include, but is not limited to, an adhesive attachment 424, sticky material, one or more mechanical attachments such as hook and loop fastener, or one or more straps 426 that can be used to wrap around a body part of a patient or a stationary object near the suture area, or a combination thereof.

In an example, the member 428 can be rotatable relative to the base 422. For instance, a rotatable coupling 432 can be disposed between the base 422 and the adhesive attachment 424. In another option, the rotatable coupling 432 can be disposed between two or more portions of the base 422, allowing the base 422 coupled with the member 428 to rotate. The rotatable member can help allow flexibility in position when the needle is picked up with the needle driver. For instance, when a simple interrupted stitch is desired, the needle of the suture assembly can be inserted and picked up in the same orientation. However, if a horizontal/vertical mattress stitch is desired, the needle can placed on a needle driver in the opposite orientation. In an example, the member 428 can be rotated about 180 degrees to allow orientation of a curved needle. Rotating the member 428 can help allow for proper orientation to load the needle driver without having to touch the needle directly. In a further option, the suture holder device can include additional structure such as to facilitate ease of rotation, for example, one or more indentations or projections such as for rotating the member 428 with a surgical tool.

In an example, the suture holder device can be combined with another device, such as a skin marker. The suture holder device can include one or more markings imprinted in ink, such as a ruler marking, and when markings are exposed, for instance to air, moisture, or on the skin of a patient, dye or ink is left on the skin, for instance, in a specified pattern or spacing. A further option is to have a releasable adhesive, so that the device can be used to first mark the skin, then expose the adhesive or other attachment, and couple the attachment near the suture area.

FIG. 4 shows another example that can include a skin marking device and a suture holder. In an example, the suture holder device 420 can be secured to a first portion of a skin marking device, such as one of the rotatable skin marking members, and an attachment optionally can be on a second portion of the skin marking device, such as another one of the rotatable skin marking members. The device can be adjusted to the appropriate distance between skin marking members, and the skin can be marked. The device can optionally be secured near a suture area, for instance by adhesively attaching the device on or near a patient. A suture holder can be used to retain suture assemblies, and the adjustment assembly can allow for the suture holder device to rotate, such as discussed above.

In an example, a method of using a suture holder device can include attaching a base of the suture holder device near a suture area, where the suture holder device can include a member coupled with the base. The member, as further described above, can have one or more suture assembly holders. The suture holder device can further include at least one attachment coupled with the base, and can further optionally include a skin marking device, which can be used to mark the skin of a patient. The method can include placing a suture assembly in the one or more suture assembly holders, and rotating the member and the one or more suture assembly holders relative to the base, for instance, but not limited to, rotating the member about 180 degrees.

Several options for the method are as follows. For instance, in an example, the method optionally includes de-coupling the member from the base prior to placing the suture assembly in the one or more suture assembly holders. In a further example, the method can further include (e.g., with any of the above) adhering the base, for example, to a suture site.

The method of using the suture holder device can further optionally include using the skin marking device, for instance, before using the suture holder device during a procedure. The method can optionally include marking the skin with the skin marking device, where a position of the skin marking members, such as two or more members, can be adjusted. In a further option, the skin marking device can include two or more opposed skin marking members, and the method can include rotating the skin marking device and rotating the two or more opposed skin marking members of the skin marking device.

In another example, the method of using the suture holder device and the skin marking device can further optionally include placing the skin marking device around a laceration and marking the skin with at least a first mark, lifting and moving the skin marking device, overlapping a first skin marking member on the at least first mark, and marking at least one additional mark with a second skin marking member while the first skin marking member overlaps the first mark.

Additional Notes

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown and described. However, the present inventors also contemplate examples in which only those elements shown and described are provided.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

1. A suture holder device, comprising:
a base;
a member, rotatably coupled with the base, the member having one or more suture assembly holders; and
at least one attachment coupled with the base, the attachment adapted to couple the suture holder device with other structure.

2. The suture holder device as recited in claim 1, wherein the member is removably coupled with the base, and the member is removable from the base.

3. The suture holder device as recited in claim 1, wherein the one or more suture assembly holders includes at least one recess in the base.

4. The suture holder device as recited in claim 1, wherein the at least one attachment includes a mechanical attachment.

5. The suture holder device as recited in claim 1, wherein the at least one attachment includes an adhesive.

6. A method of using a suture holder device, comprising:
attaching a base of the suture holder device near a suture area, the suture holder device including a member coupled with the base, the member having one or more suture assembly holders, at least one attachment coupled with the base; and
placing a suture assembly in the one or more suture assembly holders; and rotating the member and the one or more suture assembly holders relative to the base.

7. The method of using the suture holder device as recited in claim 6, further comprising de-coupling the member from the base prior to placing the suture assembly in the one or more suture assembly holders.

8. The method of using the suture holder device as recited in claim 6 , wherein attaching the base of the suture holder device includes adhering the base.

9. The method of using the suture holder device as recited in claim 6 , wherein rotating the member and the one or more suture assembly holders relative to the base includes rotating the member about 180 degrees.

10. The method of using the suture holder device as recited in claim 6 , wherein the suture holder device further includes a skin marking device, and further comprising marking skin of a patient with the skin marking device.

11. The method of using the suture holder device as recited in claim 10, further comprising adjusting a position of two or more skin marking members of the skin marking device.

12. The method of using the suture holder device as recited in claim 10, further comprising rotating the skin marking device and rotating two or more opposed skin marking members of the skin marking device.

13. The method of using the suture holder device as recited in claim 10, further comprising placing the skin marking device around a laceration and marking the skin with at least a first mark, lifting and moving the skin marking device, overlapping a first skin marking member on the at least a first mark, and marking at least one additional mark with a second skin marking member while the first skin marking member overlaps the first mark.

14. A skin marking device, comprising:
two or more skin marking members;
a holding member coupled with the two or more skin marking members;
the holding member including at least one adjustment assembly; and
the at least one adjustment assembly adjusts at least one of width or length between the skin marking members.

15. The skin marking device as recited in claim 14, wherein the two or more skin marking members are rotatable relative to the holding member.

16. The skin marking device as recited in claim 14 , further comprising at least one suture holder coupled with at least one of the two or more skin marking members, the holding member, or the adjustment assembly.

17. The skin marking device as recited in claim 14 , wherein the two or more skin marking members includes a pair of opposed rotating marking members, the opposed rotating marking members disposed along the holding member.

18. The skin marking device as recited in claim 14 , further comprising dye on or within the two or more skin marking members.

19. The skin marking device as recited in claim 14 , further comprising a numbing agent on or within the two or more skin marking members.

20. The skin marking device as recited in claim 14 , further comprising one or more elliptical skin marking members.
